(19) United States
(12) Patent Application Publication
(10) Pub. No.: US 2016/0367770 A1
(43) Pub. Date: Dec. 22, 2016

(54) ORAL INHALER MIXER SYSTEM AND METHOD

(71) Applicant: Hari Matsuda, Nevada City, CA (US)

(72) Inventor: Hari Matsuda, Nevada City, CA (US)

(21) Appl. No.: 15/184,873

(22) Filed: Jun. 16, 2016

Related U.S. Application Data
(60) Provisional application No. 62/180,194, filed on Jun. 16, 2015.

(51) Int. Cl. A61M 15/00; A61M 16/14

(52) U.S. Cl. CPC A61M 15/0086; A61M 15/0021; A61M 16/14; A61M 2207/00

(57) ABSTRACT

An add-on holding chamber for an inhaler mixer system has a holding chamber, mouthpiece, and attachment interface is coupled to the mouth inlet of the prior art inhaler, to provide a better mixing efficiency. The chemical from the medicament canister is sprayed into the air inlet side of the prior art inhaler, which enters the holding chamber, and as the user sucks onto the mouthpiece of the add-on holding chamber, air flows into the holding chamber (via inlets in the holding chamber which operate to move more air and/or vortex additional air) towards the air inlet side of the prior art inhaler. The added inhaled air provides more air and force to aerosolize with more efficiency the chemical/air mixture in the holding chamber.

### CROSS-REFERENCE TO RELATED APPLICATION(S)

This application claims the benefit of U.S. Provisional Patent Application No. 62/180,194, filed Jun. 16, 2015, the contents of which are hereby incorporated by reference in its entirety.

### FIELD

This invention is directed to holding chambers used in conjunction with oral inhalers. More particularly, to an improved mixer for an inhaler apparatus attached to a chemical or medicament containing canister.

### BACKGROUND

Inhalers with replaceable canisters are composed of a simple receptacle for attaching a medicament canister. These inhalers are very compact, having a short tube extending directly from the inhaler receptacle to the user's mouth. The inhaler is designed for mixing the medicament (or chemical) from the canister directly with external air. The inhaler can be attached to a holding chamber where the chemical is sprayed into the inhaler side of the holding chamber towards the mouthpiece. The user then creates a vacuum when sucking on the mouthpiece, drawing the chemicals to mix with external air into the user's mouth for entry into the lungs.

However, due to limitations in the physics of these holding chambers, most of the chemical is concentrated and/or settled at the mouth end of the holding chamber. Thus, the chemical is mostly in concentrated liquid droplet form at the mouth end of the inhaler, and not well mixed with incoming air while introduced into the user's mouth. When insufficient mixture occurs, the medicament or chemical is undiluted and can cause irritation or harmful effects to the user's throat and mouth, requiring the majority of inhaler users being cautioned to rinse their mouth/etc. after use.

Accordingly, in view of the very long history of the ineffectiveness of conventional inhaler apparatuses, there has been an unmet need in the industry for more efficient holding chambers. Aspects of an improve inhaler mixing system and method are detailed in the following description.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In one aspect of the disclosed embodiments, an oral inhaler holding chamber mixing device is provided, comprising: a sealed holding chamber with three openings: an external air inlet opening, an interface opening, and a mouthpiece opening; and a holding chamber-to-inhaler interface coupled to the interface opening of the holding chamber, the interface comprising: a channel disposed through the interface, adapted to provide a pathway between a top and bottom of the interface; a sealing portion, adapted to match the holding chamber's interface opening, sealing the holding chamber's interface opening but for the channel; and an attachment feature disposed on an upper portion of the interface, adapted to enable removable attachment of an inhaler mouthpiece to the interface and align a mouthpiece of an inhaler with the channel, wherein the mixing device aerosols a chemical from an attached inhaler with external air drawn through the inlet opening in the holding chamber, which is inhaled through the holding chamber's mouthpiece opening.

In another aspect of the disclosed embodiments, an oral inhaler holding chamber mixing device is provided, comprising: means for mixing chemicals drawn from an inhaler, the mixing means having an external air inlet opening, an interface opening, and a mouthpiece opening; and means for coupling the mixing means to the inhaler via the interface opening.

In yet another aspect of the disclosed embodiments, a method for forming an oral inhaler holding chamber mixing device is provided, which increases atomization of medicaments from an oral inhaler, the method comprising: forming a sealed holding chamber with three openings: an external air inlet opening, an interface opening, and a mouthpiece opening; and forming a holding chamber-to-inhaler interface coupled to the interface opening of the holding chamber, the interface formed by: forming a channel disposed through the interface, adapted to provide a pathway between a top and bottom of the interface; forming a sealing portion, adapted to match the holding chamber's interface opening, sealing the holding chamber's interface opening but for the channel; and forming an attachment feature disposed on an upper portion of the interface, adapted to enable removable attachment of an inhaler mouthpiece to the interface and align a mouthpiece of an inhaler with the channel, wherein the mixing device aerosols a chemical from an attached inhaler with external air drawn through the inlet opening in the holding chamber, which is inhaled through the holding chamber's mouthpiece opening.

### BRIEF DESCRIPTION OF THE DRAWINGS

FIG. 1 is an illustration of prior art inhaler systems with medicament canisters attached.

FIG. 2A is a perspective view of an exemplary inhaler mixer system.

FIG. 2B is a front view of the exemplary inhaler mixer system of FIG. 2A.

FIG. 2C is a side cut-away view of the exemplary inhaler mixer system of FIG. 2A.

FIG. 3 is blowup illustration showing air or aerosol movement within an exemplary device.

FIG. 4 is a close-up illustration of a coupling interface.

FIG. 5 is an illustration of an interface, holding chamber and removable bottom thereof.

FIG. 6A is an illustration of a bottom part shown in FIG. 5.

FIG. 6B is a closeup illustration of a bottom part flap.

FIG. 7A is a cut-away side illustration of another holding chamber bottom.

FIG. 7B is a blow up illustration of FIG. 7A.

FIG. 8A is an illustration of a modification of the embodiment of FIGS. 7A-B.

FIG. 8B is a blow up illustration of FIG. 8A.

FIG. 9A is a cut-away side illustration of another holding chamber bottom.

FIG. 9B is a blow up illustration of FIG. 9A.

### DETAILED DESCRIPTION

It is understood by the inventor that external air from the inhaler end of a conventional holding chamber allows the chemical/air mixture to enter the mouth/lungs with little negative pull, however, this new air does not adequately “combine” with the inhaler chemicals to cause complete aerosolizing, since the chemicals are already at the mouthpiece end. Thus, the chemicals are not sufficiently diluted by external air, and resultingly flows directly onto the inner mouth surfaces or the back of the throat. Since some chemicals are harmful to oral tissue, users are often required to rinse their mouth after using an inhaler. These difficulties in the prior art are due to the simplistic design of the mixing chamber and mouthpiece, which is restricted in the amount of air that can be introduced.

In view of the above, this application describes various exemplary embodiments that use an add-on mixer comprising a mixing holding chamber, mouthpiece, and attachment interface which is coupled to the mouth inlet of the prior art inhaler, to provide a better mixing efficiency. The chemical from the medicament canister is sprayed into the air inlet side of the prior art inhaler, which enters the holding chamber, and as the user inhales through the mouthpiece of the add-on mixer, air flows into the add-on mixer's holding chamber towards the air inlet side of the prior art inhaler. The added inhaled air provides a greater volume of air and force to aerosolize with more efficiency the chemical/air mixture in the holding chamber.

The highly aerosolized mixture (having more air) then can be drawn toward the add-on's mouthpiece into the inhaler's mouth/lungs. The mixture can be further mixed as it flows through an optional mixture bulb in the add-on's extension tube, prior to entering inhaler's windpipe/lungs. This design significantly improves the aerosolizing of chemical(s) from the medicament canister, providing a more complete chemical/air mixture for inhalation by the inhaler. Thus, un-aerosolized chemicals that were typically deposited into the back of the user's throat/tongue are now better mixed to result in the “complete” dosage of chemicals being properly inhaled by the user. Such an improvement will result in medication dosages being properly administered into the lungs rather than deposited into the patient's mouth, and also lessen the need for the patient to repeatedly administer “inefficient” dosages to him/herself. The latter result, of course, reduces the costs of purchasing chemicals/canisters for the patient.

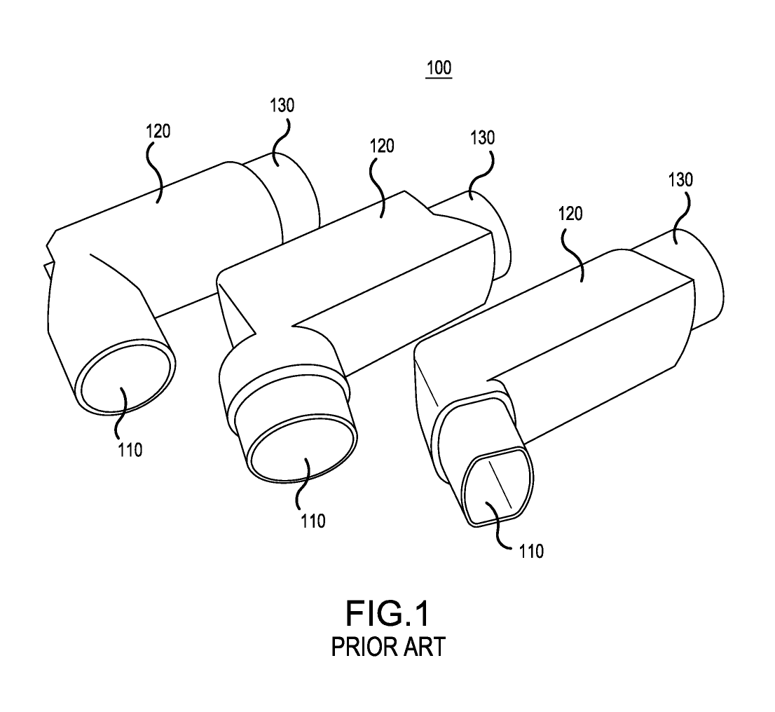

FIG. 1 is an illustration 100 showing prior art inhaler systems with medicament canisters 130 attached. The short and differently shaped inhaler mouthpieces 110 are evident for these commercial models, having outer housing 120 for the canisters 130. It is understood that these inhalers are manually operated, single dose inhalers. In some instances, the user may load up on the number of doses by multiple triggering medicament from the canister 130. In any event, regardless of the amount of medicament or chemical triggered, the user must physically with his own mouth/lungs, generate the suction to cause the medicament/chemical to be drawn into his mouth/throat.

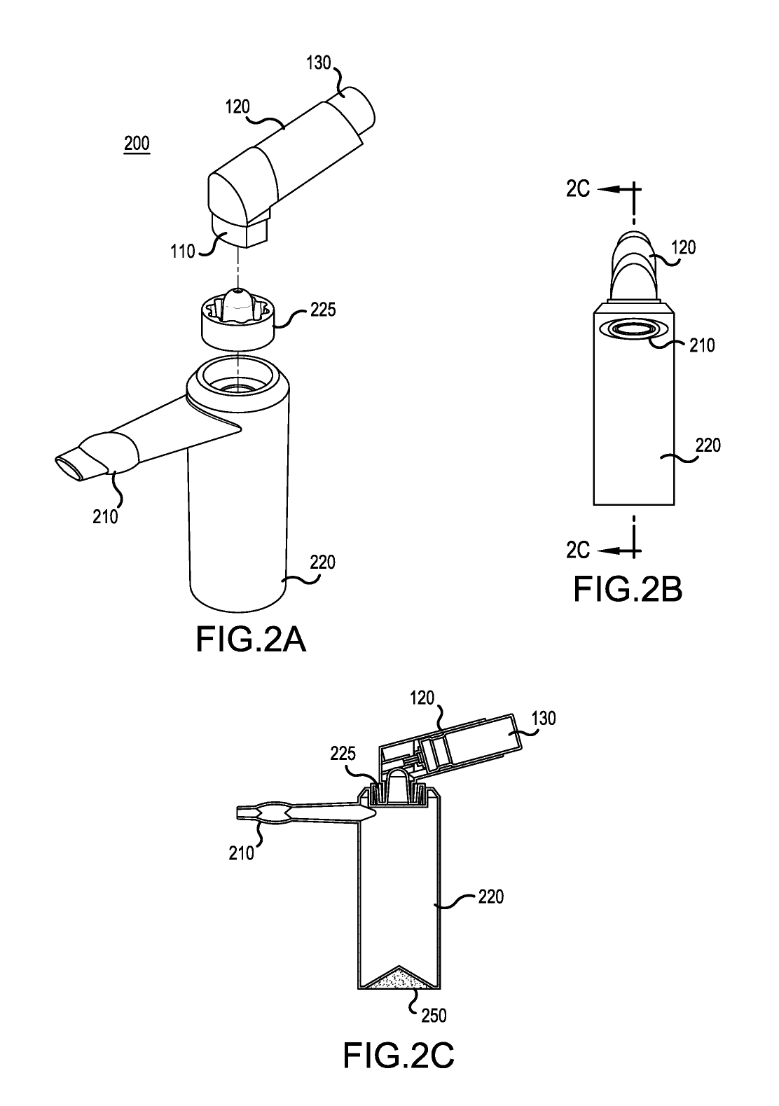

FIGS. 2A-C are perspective, front and cutaway side views, respectively, of an exemplary inhaler mixer system 200. FIG. 2A's perspective view shows a “standard” inhaler enclosure 110, 120 (as seen in FIG. 1), with accompanying medicament canister 130. The inhaler mixer system 200 is composed of a holding chamber 220 with hollow mouthpiece 210 (shown with optional secondary mixing chamber) and coupling interface 225. The interface 225 operates to “couple” the holding chamber 220 to the inhaler's mouthpiece 110. The interface 225 shown can be formed from an elastomeric material, so as to permit a frictional coupling to the inhaler's mouthpiece 110 to the holding chamber 220. Understanding that most inhaler mouthpieces are smooth-surfaced, an elastomeric or resilient coupling interface 225 obviates the need for a screw-on, snap-on, etc. coupling. However, it some embodiments, it may be desirable to have a non-elastomeric material that provides an equivalent function, depending on design preference. For example, a screw-on or latch-or other mechanism can be utilized to operatively couple the mouthpiece 110 of the inhaler to the interface 225 and to the holding chamber 220. Therefore, other methods and approaches to attaching the mouthpiece of the inhaler 110 to the interface 225 are within the purview of one of ordinary skill in the art and are understood to be within the scope of this disclosure.

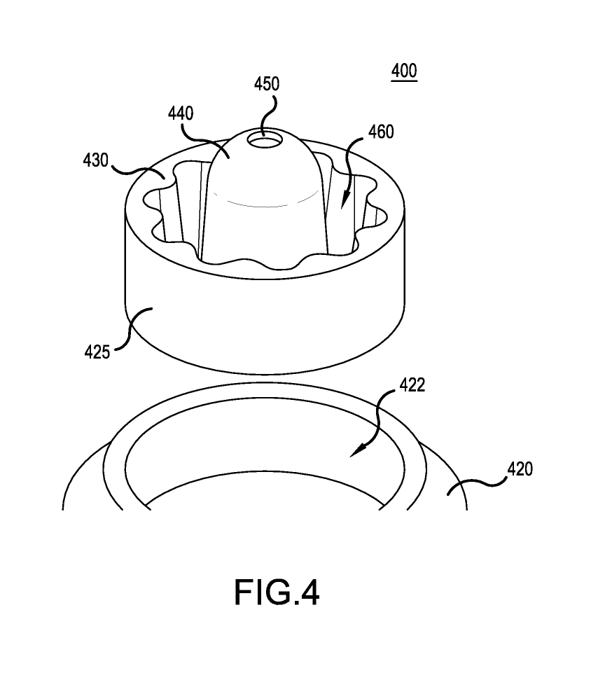

The interface 225 can be designed to mate to differently shaped mouthpieces of inhalers. For example, FIG. 2A and FIG. 2C show an elevation or side of the interface shaped with an undulating surface (described in more detail in FIG. 4). The undulations stiffen the side walls of the interface 225 to allow mating to different shapes, possibly by rotating the inhaler mouthpiece 110 until an undulation ridge or other surface is deformed to engage the mouthpiece 110, or sloped so as to permit a tilt-into action to “snap in”, or so forth. Accordingly, alternative shapes or methods may be employed to attach a prior art or other inhaler device's mouthpiece 110 to the interface 225, without departing from the spirit and scope of this disclosure.

The interface 225 serves two purposes. One, the interface 225 has a coupling mechanism that removably seals the prior art inhaler's mouthpiece 110 to the interface 225, so as to minimize or eliminate ambient air from entering at the interface-to-mouthpiece junction, while coupling the inhaler 120 to the interface 225. Two, the interface 225 guides the prior art inhaler's chemical stream from the medicament canister 130 into the holding chamber 220, so externally drawn air from the holding chamber 220 can be mixed with the chemicals.

Specifically, the interface 225 has one or more channels (see FIG. 4 for more details) to allow the inhaler's medicament/chemical to travel into the holding chamber 220. In some embodiments, the actual mixing may be accomplished within a portion of the interface 225. In other embodiments, the actual mixing may be accomplished in a portion of the holding chamber 220. In other embodiments, the actual mixing may be a combination of both. However, in the embodiments illustrated herein, the primary mixing will be accomplished in the holding chamber 220, with adjunct mixing (if desired) occurring in a subsequent bulb within the mouthpiece 210 of the exemplary mixer. Apportioning the mixing to different parts of the exemplary device is understood to be within the knowledge of one of ordinary skill in the art. Accordingly, various modifications and changes may be made to the illustrated embodiments without departing from the spirit and scope herein.

In the embodiment shown in FIG. 2A, the interface channel is centrally located within the interface 225, to align as best as possible with the main flow stream from the attached prior art inhaler. In other embodiments, the channel may be offset or changed in size or shape, allowing for angular or offset jetting of the chemicals into the holding chamber 220.

The second illustration, FIG. 2B simply illustrates a front view of the exemplary device, showing the mouthpiece opening and is self-explanatory.

FIG. 2C is a side cut-away view, showing some of the internals of the exemplary device. The holding chamber 220 is illustrated here with three orifices or openings—one for the hollow mouthpiece, another for the interface 225, and one for the ambient air inlet. It is noted the holding chamber is shown with the ambient air inlet at its lower section 250, so when the user inhales on the exemplary inhaler's mouthpiece 210, external air can be drawn into the holding chamber 220, which when balancing the different orifice sizes, also draws the chemicals deposited through the interface 225 from the prior art inhaler 120. The user may initiate mixing by inhaling and discharging the chemicals from the prior art inhaler 120, simultaneously or sequentially, as needed.

The large-sized holding chamber 220 is beneficial since it provides a higher volume and velocity of air for a given force, than via the prior art inhaler 120 by itself. The resulting higher velocity of moving air more efficiently aerosolizes the chemicals and is drawn into inhaler's mouth via the exemplary mouthpiece 210. A secondary mixing (or holding) chamber may be disposed in the mouthpiece 210, if so desired. While the secondary chamber is illustrates as spherical in shape, it is understood that it may be any desired shape. Similarly, the interface 225 may be of any desired shape.

In other embodiments, the holding chamber 220 may be inverted to be “on top” of the interface 225 and the interface's “bottom” may mate with the prior art inhaler 130. This embodiment would essentially be a flipped version of what is shown in the previous FIGS. While it is presumed the air inlet 250 for the holding chamber 220 is located at the bottom of the holding chamber 220, the air inlet 250 can be designed to be at another location, depending on holding chamber size, shape, etc. Further, more than one air inlet (and even at different portions of the holding chambers) may be used. Based on the shape/positioning/number of air inlets, a vortexing force can be introduced into the holding chamber, causing more efficient atomization/aerosolizing of the introduced chemical. In various test embodiments built by the inventor, a very high degree of aerosolizing was achieved.

In experimental versions, the approximate size of the holding chamber 220 was 5″ high by 2″ in diameter with the extension tube 3″ long and 2″ wide. The tube can be tapered to increase overall mixture velocity as the mixture enters the mixture bulb of the mouthpiece 210 and then enters the back of the user's mouth and into the throat/lungs. Since the mechanics of vaporizing or aerosolizing a fluid is within the understanding of one of ordinary skill, it is understood that various other dimensions or sizings may be contemplated without departing from the spirit and scope of this disclosure.

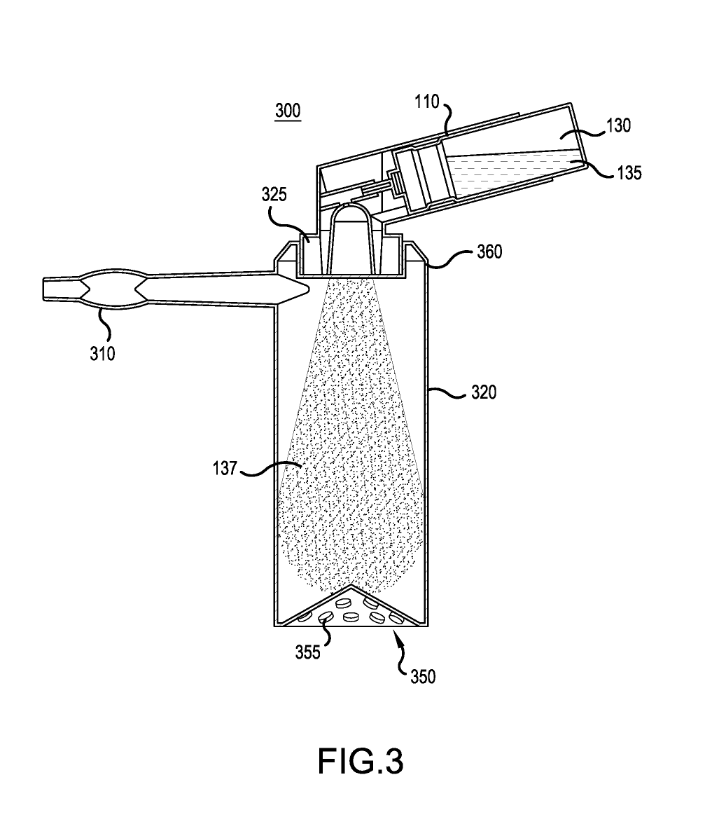

FIG. 3 is a blow-up illustration 300 showing air or aerosol movement within an exemplary device. Medicament 135 in the canister 130 is drawn into the interface 325 which mates the upper portion 360 of the holding chamber 320. Here, the aerosolizing 137 of the chemical in the holding chamber 320, is evident, being combined with external ambient air drawn in the holes 355 situated in the bottom 350 of the holding chamber 320. The aerosolized chemical 137 is then drawn into the back of the user's mouth and into the throat/lungs via mouth piece 310.

FIG. 4 is a close-up illustration 400 of a coupling interface 425. It is noted the interface 425 may, in some embodiments, be removable from the holding chamber 420. It may also be made integral to the holding chamber 420 to form a single interface-holding-chamber device, if so desired. The top of the interface 440 is shown as having a hemispherical shape with an inlet 450 allowing passage of medicament from the prior-art inhaler (not shown). The inlet 450 is shown as a simple hole, but may be tapered throughout the interface 450 or have a tapered tip. The upper shaped end 440 of the interface 425 helps minimize excess air entry into the channel's 450 entrance. However, it is understood that the hemispherical shape may be replaced with another shape, one that may better fit onto an unusually shaped inhaler spray head, for example. Or, the shape 440 may be entirely removed, leaving a channel spanning the top of the interface 425 to the bottom of the interface.

Also shown is a close-up view of one possible embodiment of the interface's undulating surface 430. The undulations 430 can be configured with different thicknesses to stiffen the side walls of the interface 425 to allow mating to different shapes, possibly by rotating a inhaler mouthpiece until an undulation ridge or other surface is deformed to engage the mouthpiece, or by a tilt-into action, or so forth. The inhaler mouthpiece would slide/fit into the circularly-shaped channel 460 inside the interface 425. Of course, the channel 460 may be of any orientation, shape or design, long as it provides some mechanism for fitting the inhaler mouthpiece end into the interface 425.

A bottom of the interface 425 may be made to be more rigid so as to mate to the top portion 422 of the holding chamber 425 below via a simple slip-in press-fit, to cause a seal, if so desired. The mechanisms for “fitting” and “sealing” the interface 425 to the top 422 of the holding chamber can be varied, for example, a screw-in or other mechanism can be used. Therefore, alternative approaches may be implemented without departing from the spirit and scope of this disclosure. For example, while the various embodiments show circular or round interfaces, canisters, holding chamber, etc., the elements may be of any desired shape that provides similar attachment and aerosolizing capability.

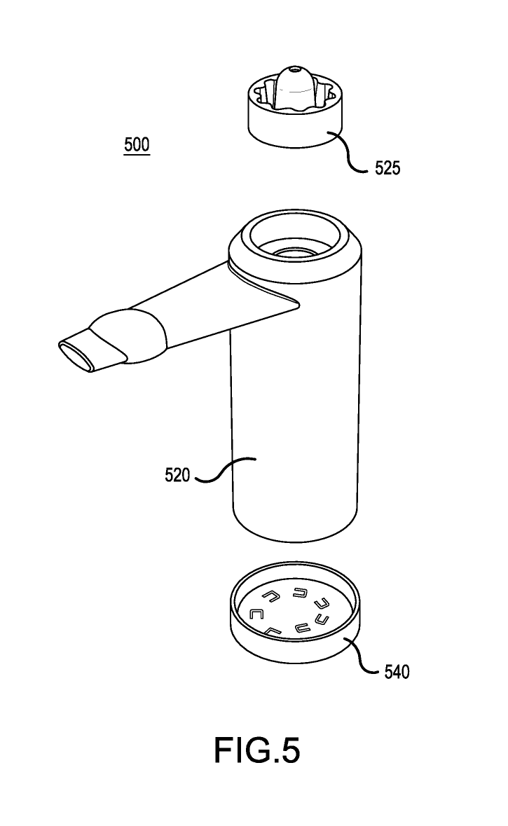

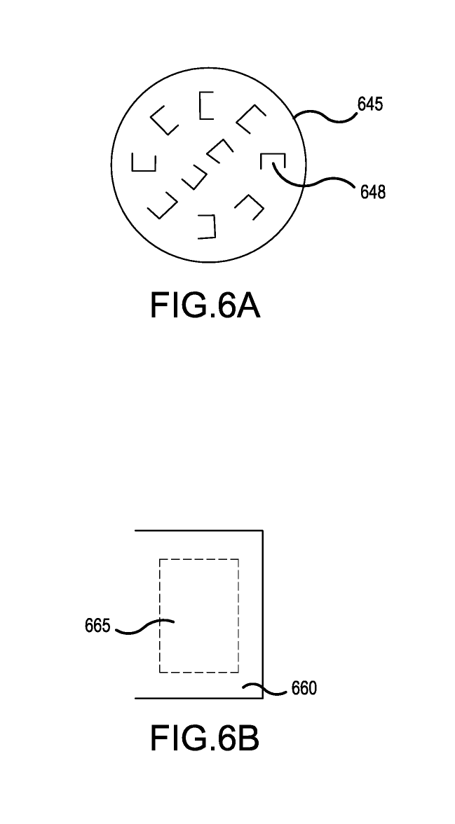

FIG. 5 is an illustration 500 of an interface 525 with a removable bottom of the holding chamber 520. This embodiment illustrates a possible molded part 540 with flaps (described in FIG. 6) that fit to an open end of the bottom of the holding chamber 520. This contemplates a design where the holding chamber's introduced air can be customized by different bottom end caps, having different sized openings. The opening can be closed via the use of flexible flaps, which permit air to enter into the bottom of the holding chamber, while preventing air from exiting out of the flaps. The flaps also minimize if not eliminate any chemical escaping through the flap holes. While FIG. 5 shows the holes at the bottom end of the holding chamber, the holding chamber can also just have small holes with flaps cut out at other sections of the holding chamber. It should be noted the flaps or openings may be of varied sized, if so desired.

FIGS. 6A and 6B are illustrations of the bottom part 540 shown in FIG. 5 and a closeup of a flap, respectively. In this illustration, the bottom part has air entrance ports for the holding chamber, which are defined by a disc 645 with U-shaped perforations 648. The “cut-outs” form flaps 660 that open over holes 665 that are smaller than the flaps 660 (or can be the same size) when air is sucked in from the mouthpiece allowing air to flow into the holding chamber. By arranging the location and orientation of the “cut-outs,” a vortex-like air flow can be generated into the holding chamber. It should be noted that while FIGS. 6A-B illustrate a U-shaped perforation, other shapes or combination of shapes, holes, etc., may be implemented, according to design preference. This design presents a customizable disc, one that can be exchanged or removed from the exemplary system. In some embodiments, the disc may not be actually shaped like a disc, but have a different geometric form, such as a square, oval, etc. Further, in some embodiments, the disc may be permanently attached to the holding chamber.

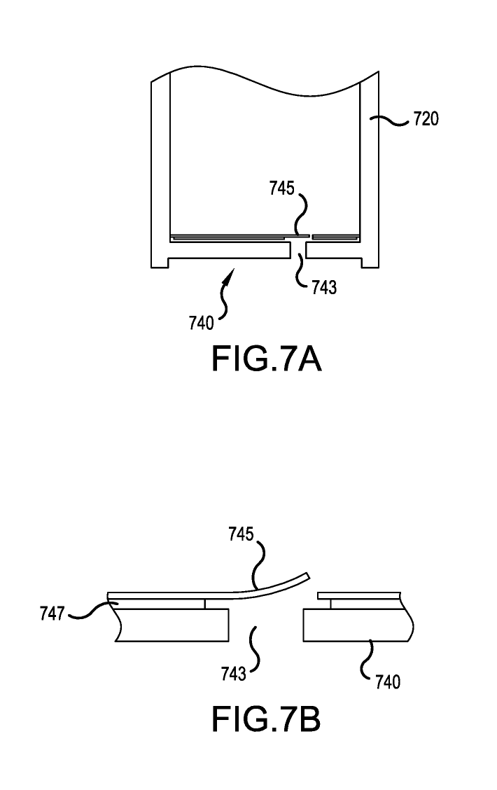

FIGS. 7A and B are a cut-away side illustration and blow up, respectively of another embodiment a holding chamber bottom with a flexible flap 745, which is glued 747 or fixed to the bottom 740 of the holding chamber 720. This design contemplates the bottom 740 of the holding chamber 720 having a pre-existing opening that is covered by a “flexible sheet” with secondary opening(s). The sheet can be of an elastomeric material or other flexible material, and can have one or more “cuts” or secondary openings that act as flaps 745. The arrangement of the flaps 745 and holding chamber opening(s) can be designed so that the air passage way 743 directs air into the holding chamber at an angle. Of course, more than one opening may be contemplated, either in the holding chamber bottom, or in the sheet. In some embodiments, the holding chamber opening may be a single large opening, and the sheet opening may be multiple openings. In other embodiments, the flexible sheet may be on the opposite side of the holding chamber surface.

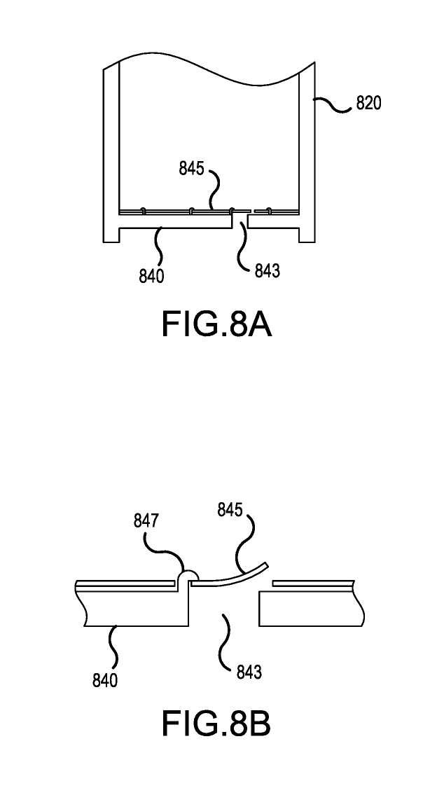

FIGS. 8A and B are illustrations of a modification of the embodiment of FIGS. 7A-B, showing the flaps 845 being anchored to “hooks” 847 at the bottom 840 of the holding chamber 820 and is demonstrative of various other approaches that may be employed to allow/control the entrance of air thorough air channels 843 in the holding chamber 820.

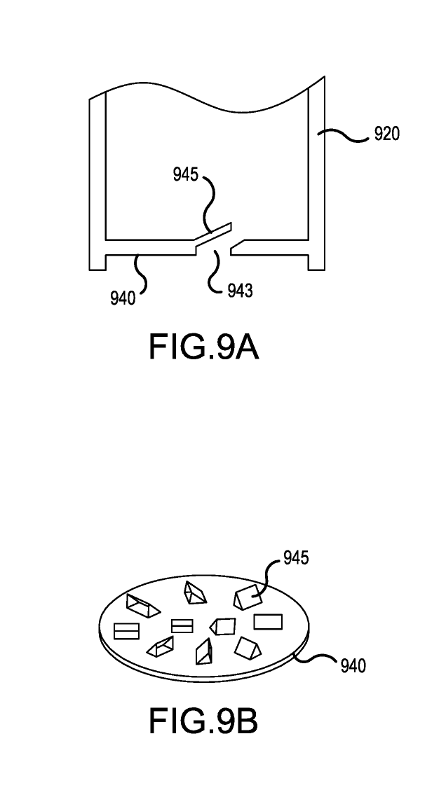

FIGS. 9A and B are illustrations of another possible configuration for the holding chamber's “bottom” openings, comprising a series of molded vents 945 having the air inlet 943 being directed at an angle. The bottom openings can be pre-made into the holding chamber 920 or be part of a disc 940 that is attached to the bottom end of the holding chamber 920. In some embodiments, the surfaces of the inlet boundaries can be chamfered or angled to minimize turbulence. It should be appreciated that the vents or opening minimize the possibility of chemicals exiting via the bottom of the holding chamber 920.

Alternative embodiments are envisioned for the designs shown in the above FIGS. For example, a combination mouthpiece and interface of a single integrated piece is contemplated, where the holding chamber is a wholly separate device, one that has different sizes or air inlet sizes/locations. This will allow a user to vary the “efficiency” or characteristic of a holding chamber that is to his preference, while using the same combination mouthpiece and interface. In other embodiments, the mouthpiece and holding chamber may be separate, but attachable to each other. This allows a certain degree of customization for a user, not to mention affords the ability to make the device more compact, if in separate pieces. In some embodiments, an adjustable opening can be used for the holding chamber's air inlet. This would allow the user to adjust the amount of air used in the mixing.

It should be appreciated that while the terms elastomeric and molded, etc. are used in the FIGS., they are not limiting terms but only describe a possible mode of quality or fabrication. Other qualities, fabrication, materials may be used, as desired. For example, rubber, or silicone. Also, the holding chamber may be made of a flexible material, one that collapses for easier storage.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. An oral inhaler holding chamber mixing device, comprising:
a sealed holding chamber with three openings: an external air inlet opening, an interface opening, and a mouthpiece opening; and
a holding chamber-to-inhaler interface coupled to the interface opening of the holding chamber, the interface comprising:
a channel disposed through the interface, adapted to provide a pathway between a top and bottom of the interface;
a sealing portion, adapted to match the holding chamber's interface opening, sealing the holding chamber's interface opening but for the channel; and
an attachment feature disposed on an upper portion of the interface, adapted to enable removable attachment of an inhaler mouthpiece to the interface and align a mouthpiece of an inhaler with the channel,
wherein the mixing device aerosols a chemical from an attached inhaler with external air drawn through the inlet opening in the holding chamber, which is inhaled through the holding chamber's mouthpiece opening.

2. The inhaler mixing device of claim 1, wherein the inlet opening is at the bottom of the holding chamber.

3. The inhaler mixing device of claim 1, wherein the inlet opening is a series of holes in the holding chamber.

4. The inhaler mixing device of claim 3, wherein the series of holes are covered by angled vents, the angled vents being interior to the holding chamber.

5. The inhaler mixing device of claim 3, wherein the series of holes are formed in a removable plate or disc that is attachable to the holding chamber.

6. The inhaler mixing device of claim 5, wherein a portion of the plate or disc is made of a flexible material, the holes being formed therein.

7. The inhaler mixing device of claim 3, wherein the holes are formed from U-shaped perforations.

8. The inhaler mixing device of claim 6, wherein the holes are covered by the flexible material, the flexible material forming flaps which are opened when air is drawn through the chamber's mouthpiece opening by the user.

9. The inhaler mixing device of claim 1, further comprising a mixing chamber within the chamber's mouthpiece opening.

10. The inhaler device of claim 1, wherein the interface is integral to the holding chamber.

11. The inhaler mixing device of claim 1, wherein the interface's attachment feature comprise undulations that grip an inhaler mouthpiece of one or more differently shaped varieties of commercial inhalers.

12. The inhaler mixing device of claim 1, wherein the holding chamber is approximately 5″ high by 2″ in diameter with a mouthpiece extension tube approximately 3″ long and 2″ wide.

13. The inhaler mixing device of claim 12, wherein the extension tube and holding chamber are a single integral piece.

14. The inhaler mixing device of claim 1, further comprising an inhaler attached to the interface.

15. The inhaler mixing device of claim 1, further comprising a chemical-containing canister attached to the inhaler.

16. An oral inhaler holding chamber mixing device, comprising:
means for mixing chemicals drawn from an inhaler, the mixing means having an external air inlet opening, an interface opening, and a mouthpiece opening; and
means for coupling the mixing means to the inhaler via the interface opening.

17. The inhaler mixing device of claim 16, wherein the mixing means contains openings in its bottom.

18. The inhaler mixing device of claim 17, wherein the openings are formed in a removable plate or disc.

19. An method for forming an oral inhaler holding chamber mixing device, which increased atomization of medicaments from an oral inhaler, comprising:
forming a sealed holding chamber with three openings: an external air inlet opening, an interface opening, and a mouthpiece opening; and
forming a holding chamber-to-inhaler interface coupled to the interface opening of the holding chamber, the interface formed by:
forming a channel disposed through the interface, adapted to provide a pathway between a top and bottom of the interface;
forming a sealing portion, adapted to match the holding chamber's interface opening, sealing the holding chamber's interface opening but for the channel; and
forming an attachment feature disposed on an upper portion of the interface, adapted to enable removable attachment of an inhaler mouthpiece to the interface and align a mouthpiece of an inhaler with the channel,
wherein the mixing device aerosols a chemical from an attached inhaler with external air drawn through the inlet opening in the holding chamber, which is inhaled through the holding chamber's mouthpiece opening.
